(19) United States
(12) Patent Application Publication
(10) Pub. No.: US 2014/0302577 A1
(43) Pub. Date: Oct. 9, 2014

(54) ENHANCED YEAST FERMENTATION PLATFORM USING YEAST LACKING MITOCHONDRIAL DNA AND CONTAINING GROWTH IMPROVING MUTATIONS

(71) Applicant: University of Wyoming, Laramie, WY (US)

(72) Inventors: Peter E. Thorsness, Laramie, WY (US); Christopher P. Smith, Parker, CO (US)

(73) Assignee: UNIVERSITY OF WYOMING, Laramie, WY (US)

(21) Appl. No.: 14/243,410

(22) Filed: Apr. 2, 2014

Related U.S. Application Data
(60) Provisional application No. 61/808,116, filed on Apr. 3, 2013.

(51) Int. Cl. C12N 15/81; C12P 7/06

(52) U.S. Cl. CPC C12N 15/81; C12P 7/06
USPC 435/161; 435/454; 435/254.21; 435/320.1

(57) ABSTRACT

Methods for enhanced yeast fermentation of plant material through the genetic modification of non-respiring yeast are provided including the introduction of a dominant mitochondrial ATP synthase gene mutation into a non-respiring yeast that entirely lacks mitochondrial DNA and transgenic yeast for the enhanced yeast fermentation of plant material lacking mitochondrial DNA while having a dominant mitochondrial ATP synthase gene mutation in the nuclear genome. Methods further include the introduction of a mitochondrial genome into a non-respiring yeast lacking the COX1, COX2, COX3, or COB gene as well as transgenic yeast having a mitochondrial genome lacking the COX1, COX2, COX3, or COB gene. Additional methods include the creation of a disrupted copy of the CAT5 nuclear gene in a non-respiring yeast as well as transgenic yeast having a disrupted copy of the CAT5 nuclear gene are also disclosed.

### CROSS REFERENCE TO RELATED APPLICATIONS

The present application claims priority of U.S. Provisional Patent Application Ser. No. 61/808,116, filed on Apr. 3, 2013, entitled “ENHANCED YEAST FERMENTATION PLATFORM USING YEAST LACKING MITOCHONDRIAL DNA AND CONTAINING GROWTH IMPROVING MUTATIONS,” the entire contents are herein incorporated by reference for all it teaches and discloses.

### ACKNOWLEDGEMENT OF FEDERAL RESEARCH SUPPORT

This invention was made, in part, with government support awarded by the National Institutes of Health grant # GM068066. Accordingly, the United States government has certain rights in this invention.

### SUBMISSION OF SEQUENCE LISTING

The Sequence Listing associated with this application is filed in electronic format via EFS-Web and is hereby incorporated by reference into the specification in its entirety.

### BACKGROUND

All publications cited in this application are herein incorporated by reference.

Most commercial or industrial yeast are of the species Saccharomyces cerevisiae and are capable of growth on non-fermentable carbon sources and thus contain an intact mitochondrial genome (termed petite positive). Petite positive yeast like S. cerevisiae are able to grow on a fermentable carbon source in the absence of mitochondrial DNA (mtDNA).

The foregoing examples of related art and limitations related therewith are intended to be illustrative and not exclusive, and they do not imply any limitations on the inventions described herein. Other limitations of the related art will become apparent to those skilled in the art upon a reading of the specification and a study of the drawings.

### SUMMARY

The following embodiments and aspects thereof are described and illustrated in conjunction with systems, tools and methods, which are meant to be exemplary and illustrative, not limiting in scope.

An embodiment of the present invention may comprise a method for enhanced fermentation of plant material through the genetic modification of non-respiring yeast comprising removing a mitochondrial gene from mitochondrial DNA of the non-respiring yeast, while leaving the rest of the mitochondrial genome of the non-respiring yeast intact, wherein the removed mitochondrial gene is a COX1, COX2, COX3, or COB gene and producing a non-respiring yeast where the non-respiring yeast is capable of fermenting plant material.

An embodiment of the present invention may comprise a transgenic non-respiring yeast having a mitochondrial gene removed from the mitochondrial DNA of the yeast while leaving the rest of the yeast mitochondrial genome intact, wherein the removed mitochondrial gene is a COX1, COX2, COX3, or COB gene.

An embodiment of the present invention may comprise a method for enhanced fermentation of plant material through the genetic modification of non-respiring yeast comprising: introducing a dominant mitochondrial ATP synthase gene mutation into the yeast and removing a mitochondrial gene from mitochondrial DNA of the non-respiring yeast while leaving the rest of the mitochondrial genome of the non-respiring yeast intact, wherein the removed mitochondrial gene is COX1, COX2, COX3, or COB gene.

An embodiment of the present invention may comprise a transgenic non-respiring yeast comprising a dominant mitochondrial ATP synthase gene mutation and a mitochondrial genome lacking the COX1, COX2, COX3, or COB gene.

An embodiment of the present invention may comprise a method for enhanced fermentation of plant material through the genetic modification of non-respiring yeast comprising: introducing a dominant mitochondrial ATP synthase gene mutation into the yeast.

An embodiment of the present invention may comprise a DNA construct for enhanced yeast fermentation of plant material through the genetic modification of non-respiring yeast wherein the construct comprises: a dominant mitochondrial ATP synthase gene mutation and a selectable marker, wherein the dominant mitochondrial ATP synthase gene mutation is operably linked to the selectable marker.

An embodiment of the present invention may comprise a transgenic non-respiring yeast having a DNA construct stably integrated into the transgenic non-respiring yeast under conditions suitable for expression of the DNA construct in transgenic non-respiring yeast, wherein the DNA construct comprises a dominant mitochondrial ATP synthase gene mutation and a selectable marker.

An embodiment of the present invention may comprise a method for enhanced fermentation of plant material through the genetic modification of non-respiring yeast comprising deleting a CAT5 nuclear gene of the non-respiring yeast but leaving the mitochondrial genome of the non-respiring yeast intact.

An embodiment of the present invention may comprise a transgenic non-respiring yeast having a CAT5 nuclear gene of the non-respiring yeast deleted from the yeast mitochondrial genome but leaving the mitochondrial genome of the non-respiring yeast intact.

An embodiment of the present invention may comprise a method for enhanced fermentation of plant material through the genetic modification of non-respiring yeast comprising: introducing a dominant mitochondrial ATP synthase gene mutation into the yeast and destroying a CAT5 nuclear gene of the yeast.

An embodiment of the present invention may comprise a transgenic non-respiring yeast comprising a dominant mitochondrial ATP synthase gene mutation and lacking a CAT5 nuclear gene.

In addition to the example, aspects and embodiments described above, further aspects and embodiments will become apparent by reference to the drawings and by study of the following descriptions, any one or all of which are within the embodiments of the invention. The summary above is a list of example implementations, not a limiting statement of the scope of the embodiments of the invention.

### BRIEF DESCRIPTION OF THE FIGURES

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 is a flow diagram of a method to create a yeast capable of enhanced fermentation through introduction of a dominant mitochondrial ATP synthase gene.

FIG. 2 is a flow diagram showing a method to create a yeast capable of enhanced fermentation through introduction of a mitochondrial genome lacking the COX1, COX2, COX3, or COB gene.

FIG. 3 is a flow diagram showing a method to create a yeast capable of enhanced fermentation through destruction of the CAT5 gene.

FIG. 4a is a DNA construct with the dominant ATP1-111 allele operably linked to a dominant selectable marker.

FIG. 4b is a second DNA construct with the dominant ATP1-111 allele operably linked to a dominant selectable marker.

FIG. 5 is the DNA construct of FIG. 4b cloned into an E. coli vector.

FIG. 6a, FIG. 6b and FIG. 6c provide a flow diagram showing removal of selectable marker located adjacent to the dominant ATP1-111 allele.

FIG. 7 shows a diagram of a yeast DNA sequence showing the complexity of creating an ATP1 gene construct with a selectable marker due to the close proximity of flanking sequences.

FIG. 8a and FIG. 8b shows two graphs showing increased F₁-ATPase activity in mitochondria isolated from yeast bearing dominant mitochondrial ATP synthase gene mutations.

FIG. 9 shows six graphs showing generation of an inner mitochondrial membrane potential in ρ⁺ and ρ° yeast by addition of ATP.

FIG. 10 shows ethanol production in yeast during batch fermentation comparing wild type yeast with transgenic yeast of the present disclosure.

FIG. 11a and FIG. 11b plot of the data contained within Table 3, showing the growth rate and corresponding ethanol production in yeast during batch fermentation for wild type yeast strain (Rho+), yeast lacking mitochondrial DNA (Rho0), yeast lacking mitochondrial DNA and bearing a dominant mitochondrial ATP synthase gene mutation (ATP1-111 Rho0), and yeast bearing mitochondrial DNA lacking the COX3 gene (Rho+Mit−).

### BRIEF DESCRIPTION OF THE SEQUENCE LISTINGS

SEQ ID NO:1 discloses the ATP1-111 protein sequence with an amino acid change of Val to Phe at location 111.

SEQ ID NO:2 discloses the ATP1-111 nucleic acid sequence of ORF with G to T change at location 331.

SEQ ID NO:3 discloses the DNA sequence immediately 5′ to the ATP1 open reading frame.

SEQ ID NO:4 discloses the DNA sequence immediately 3′ to the ATP1 open reading frame.

SEQ ID NO:5 discloses the repetitive DNA sequence element used in the construction of a construct containing a dominant genetic marker such as KAN-MX.

SEQ ID NO:6 discloses the forward PCR primer for ATP1 sequencing.

SEQ ID NO:7 discloses the reverse PCR primer for ATP1 sequencing.

SEQ ID NO:8 discloses the forward PCR primer for cloning ATP1 alleles.

SEQ ID NO:9 discloses the reverse PCR primer for cloning ATP1 alleles.

### DETAILED DESCRIPTION

Embodiments of the present disclosure include methods for enhancing yeast fermentation of plant material through the genetic modification of non-respiring yeast, where the term “yeast” includes but is not limited to Saccharomyces cerevisiae. The methods for enhancing fermentation of yeast described in the present disclosure proceed through the alteration of yeast nuclear or mitochondrial genes required for growth on non-fermentable carbon sources (such as a disrupted copy of the CAT5 gene, dominant mutations in mitochondrial ATP synthase genes, or a mitochondrial genome lacking the COX1, COX2, COX3, or COB gene). The transgenic yeast of the present disclosure having a nonfunctional or absent mitochondrial DNA express enhanced fermentation and improved growth because the yeast are unable to invoke respiratory pathways, consequently preventing the metabolism or consumption of desirable fermentation intermediates or products (such as pyruvate and ethanol, respectively) and preventing significant growth defects in the transgenic yeast that preclude their use for commercial purposes, thereby increasing yeast ethanol production and yields (approximately 25% or more).

One or more embodiments of the present invention include methods for increasing the ethanol production of non-respiring yeast by providing methods for enhancing the fermentation of plant material by non-respiring yeast. One or more of these methods may include stably introducing a construct into a transgenic non-respiring yeast comprising a dominant mutation in a gene or genes encoding the mitochondrial ATP synthase such as the dominant ATP1-111 mutation of SEQ ID NO:1 or SEQ ID NO: 2. Additional embodiments may comprise a transgenic non-respiring yeast having the DNA construct for the expression of a dominant mitochondrial ATP synthase gene mutation stably integrated into the yeast's genome under conditions suitable for the expression of a dominant mitochondrial ATP synthase gene mutation. Further embodiments for increasing ethanol production may include integration of a dominant mitochondrial ATP synthase gene mutation into the yeast's genome under conditions suitable for the expression of a dominant mitochondrial ATP synthase gene mutation, where, as will be discussed in more detail later, the transgenic yeast with the dominant mitochondrial ATP synthase gene mutation also has a mitochondrial genome lacking the COX1, COX2, COX3, or COB gene or a disrupted a copy of the CAT5 gene from the transgenic non-respiring yeast's nuclear genome. As used herein, the term “expression” includes the process by which information from a gene is used in the synthesis of a functional gene product.

One or more embodiments of the present disclosure for increasing yeast ethanol production include a method comprising removing the intact mitochondrial genome from a non-respiring yeast containing the COX1, COX2, COX3, or COB gene and then introducing a new mitochondrial genome back into the non-respiring yeast where the new mitochondrial genome lack the COX1, COX2, COX3, or COB gene. Additional embodiments may comprise a transgenic non-respiring yeast produced from this method where the yeast mitochondrial genome lacks the COX1, COX2, COX3, or COB gene.

Another embodiment of the present invention includes another method for enhancing non-respiring yeast fermentation of plant material comprising disrupting a copy of the CAT5 gene from the transgenic non-respiring yeast's nuclear genome. An embodiment may further comprise a transgenic non-respiring yeast, having a disrupted copy of the CAT5 gene.

Mitochondria contain a small genome (mtDNA) encoding a subset of mitochondrially localized proteins. The mitochondrial genome is 75-85 kb in size in Saccharomyces cerevisiae (yeast) and encodes the mitochondrial ribosomal protein Var1, tRNAs, rRNAs, four cytochrome oxidase subunits that are part of the electron transport chain (COX1, COX2, COX3, and COB) and three subunits of the proton-translocating F₀ portion of the F₁F₀-ATPase (Atp6, Atp8, and Atp9) (see Smith et al, Genetics 179: 1285-1299 (2008)). Yeast strains with intact, fully-functional mtDNA (ρ+ strains) can be converted into strains without mtDNA (ρ° strains) or with dysfunctional mtDNA (ρ− strains) by inclusion of ethidium bromide (EtBr) in the growth media. Because S. cerevisiae mtDNA encodes subunits of electron transport complexes and the Fo component of ATP synthase, no electron transport or oxidative phosphorylation is possible in ρ° or ρ− strains. Yeast is considered a petite-positive organism because it is able to grow without mtDNA (ρ°) or with a mitochondrial genome severely compromised by extensive deletions (ρ−). Because four subunits of the electron transport chain and three subunits of the F₀ portion of the F₁F₀-ATPase are encoded by mtDNA, yeast lacking a mitochondrial genome must maintain membrane potential (Δψ_{M}) by exchange of ATP⁴⁻ for ADP³⁻ through the ADP/ATP carrier. ADP³⁻ is provided by the hydrolysis of ATP⁴⁻, catalyzed by the remaining F₁ portion of the ATPase (F¹⁻ ATPase).

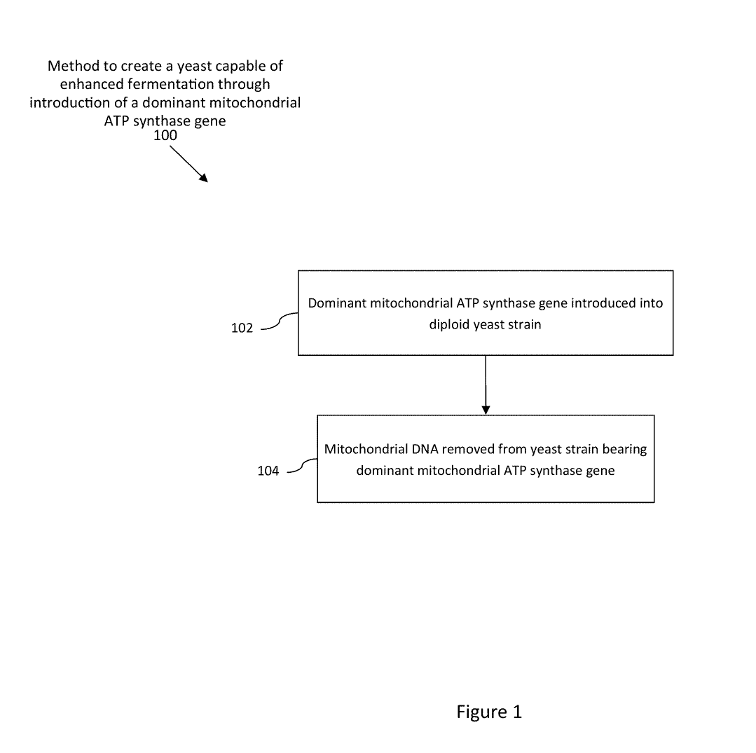

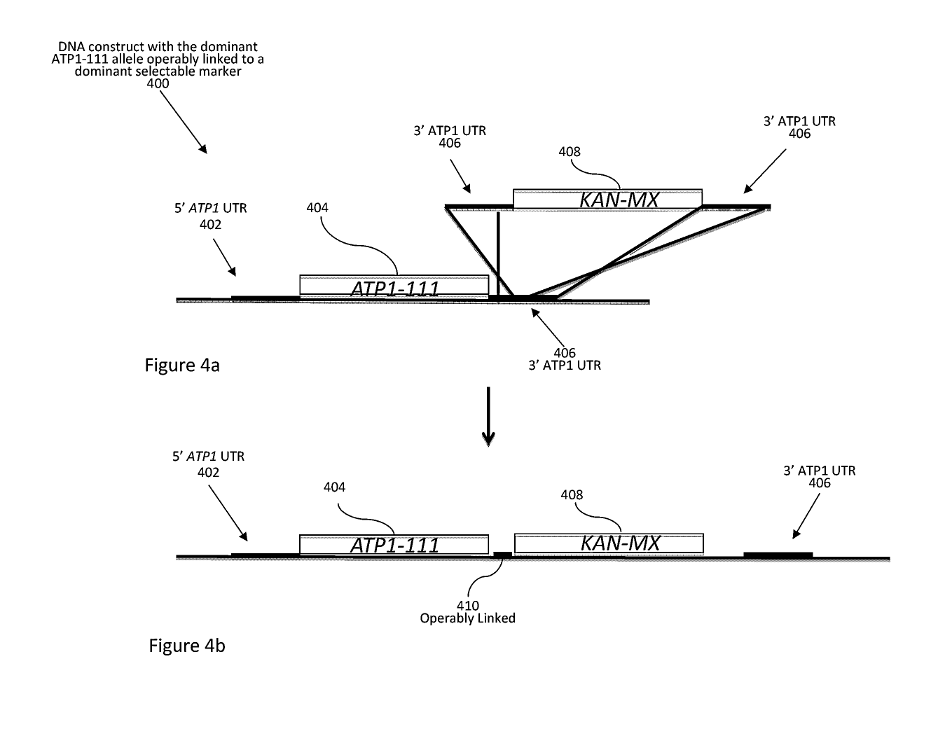

As shown in FIG. 1, a method for the enhancement of non-respiring yeast fermentation through the incorporation of a dominant mitochondrial ATP synthase gene mutation is provided, 100. As shown in FIG. 1, in step 102, a diploid non-respiring strain is transformed with a DNA construct comprising a dominant mitochondrial ATP synthase gene mutation, such as the dominant ATP1-111 mutation of SEQ ID NO:1 or SEQ ID NO: 2, and a selectable marker, where the DNA construct is introduced into the yeast nuclear genome. The construction of the DNA construct will be discussed further in relation to FIGS. 4a, 4b, 5, 6a, 6b and 6c, however standard genetic techniques may be used to introduce the ATP1-111 mutation into the yeast genome (see Sherman et al. Methods in yeast genetics. Cold Spring Harbor, N.Y., Cold Spring Harbor Laboratory Press (1986)). In step 104, diploid yeast bearing the dominant mitochondrial ATP synthase gene mutation are grown in the presence of ethidium bromide to induce the loss of mitochondrial DNA, rendering the yeast cells incapable of growth on non-fermentable carbon sources. As will be discussed in more detail, the metabolic change in the transgenic yeast of the present disclosure enhances the membrane potential and increases the ATPase activity of the F₁ portion of the mitochondrial ATP synthase. These modified industrial yeast strains lacking mitochondrial DNA entirely but containing a dominant mitochondrial ATP synthase gene mutation are incapable of respiration but maintain a mitochondrial membrane potential of sufficient magnitude to support vigorous cell growth and enhanced fermentation properties.

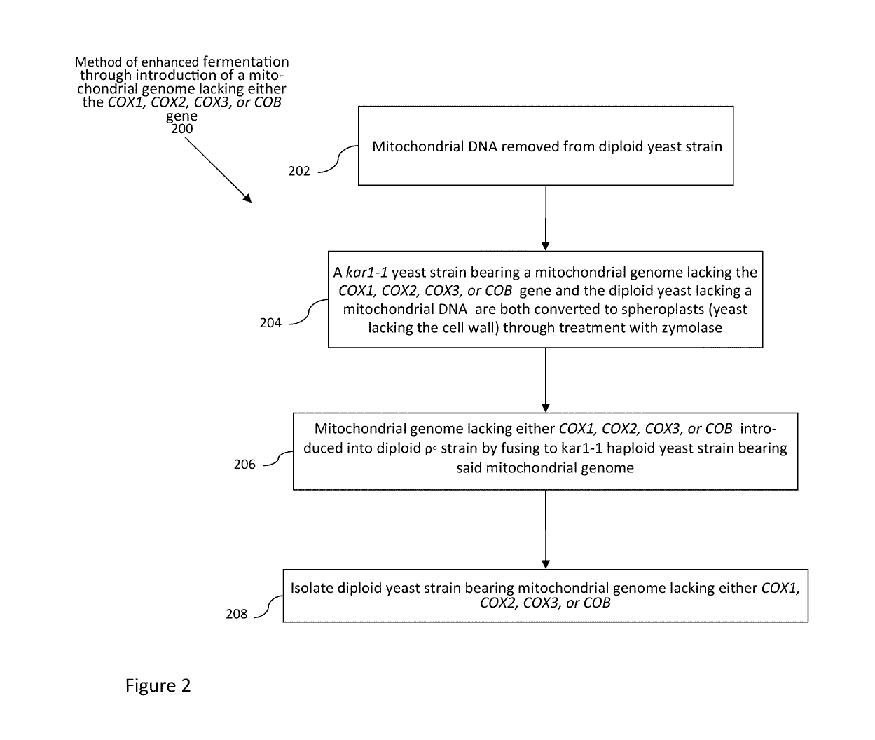

In FIG. 2, a flow chart is provided showing the steps of the introduction of a mitochondrial genome lacking the COX1, COX2, COX3, or COB gene into a diploid yeast cell, 200. As shown in FIG. 2, in step 202, the mitochondrial DNA of an industrial strain diploid yeast is removed from the diploid yeast by growing the diploid yeast in the presence of ethidium bromide. In step 204, using conventional conversion techniques, a kar1-1 yeast strain bearing a mitochondrial genome lacking the COX1, COX2, COX3, or COB gene and the industrial diploid yeast of step 202 are both converted to spheroplasts (yeast lacking the cell wall) through treatment with zymolase, a lytic enzyme used in digestion, in osmotically supportive media. In step 206 the kar1-1 yeast strain bearing a mitochondrial genome lacking the COX1, COX2, COX3, or COB gene is fused in the presence of 10 mil/CaCl₂ and 40% w/v polyethlene glycol to the industrial diploid yeast strain lacking mitochondrial DNA of step 202. The spheroplasted kar1-1 yeast bearing the mitochondrial genome lacking the COX1, COX2, COX3, or COB gene is incubated at a fifty to one hundred-fold excess with the spheroplasted industrial diploid yeast lacking mitochondrial DNA. In step 208, fused yeast are selected that contain only the nuclear genome of the industrial diploid strain and the mitochondrial genome lacking the COX1, COX2, COX3, or COB gene. The fused spheroplasted yeast were regenerated in osmotically supportive media containing 1.2 M sorbitol. These modified industrial yeast strains are evidenced by the recovery of fast growing prototrophic yeast. The nuclear genetic integrity of the recovered diploid yeast is examined by PCR of a nuclear locus that is heterozygous with respect the haploid strain that provided the mitochondria genome lacking the COX1, COX2, COX3, or COB genes. Such heterozygous nuclear loci include but are not limited to regions corresponding to TRP1, LYS2, ADE2 and URA3. The mitochondrial genome structure (e.g.—the lack of the COX1, COX2, COX3, or COB gene) is verified by PCR using oligonucleotides that span the deleted region of the mitochondrial genome. These modified industrial yeast strains bearing a mitochondrial genome lacking the COX1, COX2, COX3, or COB genes, are incapable of respiration but maintain a mitochondrial membrane potential of sufficient magnitude to support vigorous cell growth, with doubling times equal to the doubling time of yeast bearing a mitochondrial genome with the COX1, COX2, COX3, or COB genes. The modified industrial yeast strains bearing a mitochondrial genome lacking the COX1, COX2, COX3, or COB genes also have enhanced fermentation properties that allows for increased ethanol production (between 5% and 25% greater ethanol production than yeast bearing an intact mitochondrial genome). The method of FIG. 2 may be combined with the method of FIG. 1 (without the removal of the mitochondrial DNA of step 104) to create modified industrial yeast strains bearing a dominant mitochondrial ATP synthase gene mutation and also possessing a mitochondrial genome lacking the COX1, COX2, COX3, or COB genes. All DNA manipulations are performed using standard techniques (see Sambrook, et al., Molecular Cloning, 2nd Ed., Cold Spring Harbor Laboratory Press, Cold Spring Harbor, N.Y. (1989)). Restriction and DNA modification enzymes were purchased from New England Biolabs unless otherwise noted.

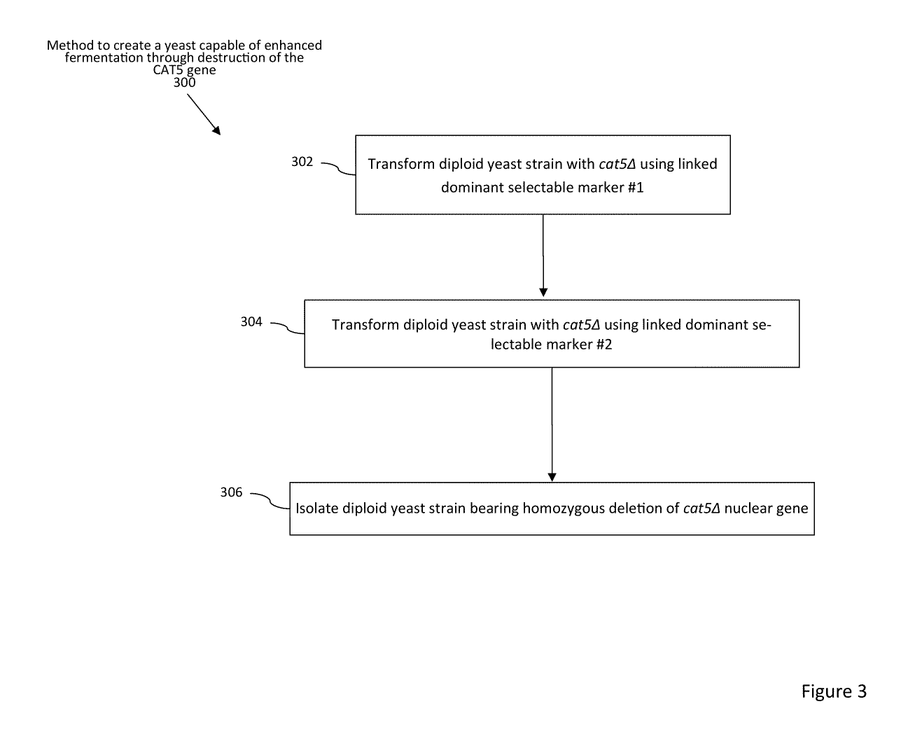

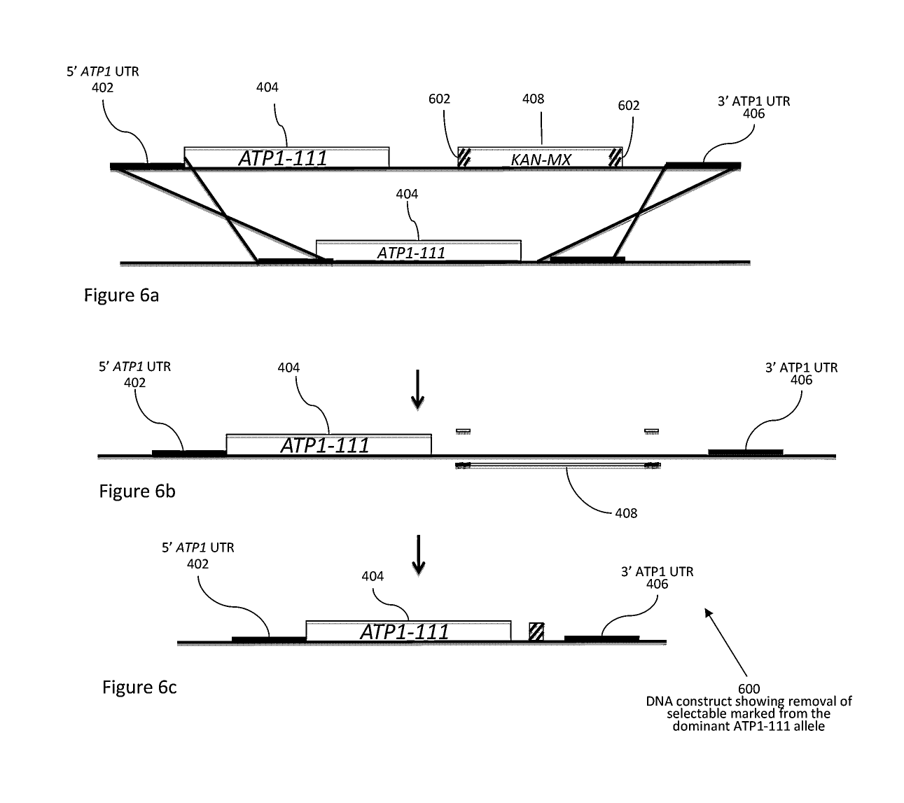

In FIG. 3, a flow chart is provided for a modified diploid yeast strain that is unable to respire (grow on non-fermentable carbon sources) but has vigorous growth is made by deleting a nuclear gene, such as the CAT5 gene, necessary for a functional mitochondrial electron transport chain 300. Deletion of nuclear mutations of this class leave an intact mitochondrial ATP synthase and allows the formation of a mitochondrial membrane potential that supports vigorous growth. FIG. 3 uses the nuclear gene CAT5 as an illustrative example. As shown in FIG. 3, because industrial yeast are diploid, to facility the deletion of the nuclear gene, two successive transformations are made using DNA constructs that disrupt the two copies of the nuclear CAT5 gene (the disrupted allele is indicated as “cat5A”) using different dominant selectable markers. The DNA constructs are standard DNA constructs comprising linear DNA fragments with homology to the 5′ and 3′ ends of the CAT5 gene, however the middle of CAT5 gene is replaced by the dominant selectable marker, such as a drug resistance gene. The DNA construct may be created using conventional genetic techniques such as the genetic technique described in FIG. 6, where repeated sequences flanking sequences of the selectable marker where the directly repeated sequences undergo frequent spontaneous recombination, leading to loss of the selectable marker sequence. In step 302, the first integrative transformation will delete the first of the two copies of the CAT5 gene. In step 304, the second integrative transformation will delete the second copy of the nuclear gene. In step 306, yeast strains bearing the cat5Δ deletion are isolated. The method of FIG. 3 may be combined with the method of FIG. 1 (without the removal of the mitochondrial DNA of step 104) to create modified industrial yeast strains bearing a dominant mitochondrial ATP synthase gene mutation incapable of respiration that also bears the cat5Δ deletion. Modified yeast strains bearing homozygous deletion of cat5Δ nuclear genes are expected to have vigorous cell growth, with doubling times equal to the doubling time of yeast bearing an intact CAT5 nuclear gene and further are expected to have enhanced fermentation properties that allows for increased ethanol production between 5% and 25% greater than unmodified yeast bearing an intact CAT5 nuclear gene. The method of FIG. 3 may be combined with the method of FIG. 1 (without the removal of the mitochondrial DNA of step 104) to create modified industrial yeast strains bearing a dominant mitochondrial ATP synthase gene mutation and also possessing a homozygous deletion of cat5Δ nuclear gene. All DNA manipulations are performed using standard techniques (see Sambrook, et al., Molecular Cloning, 2nd Ed., Cold Spring Harbor Laboratory Press, Cold Spring Harbor, N.Y. (1989)). Restriction and DNA modification enzymes were purchased from New England Biolabs unless otherwise noted.

Saccharomyces cerevisiae is routinely used in ethanolic fermentations that produce biofuels. This yeast is capable of fermentation of hexoses, such as glucose, to ethanol in the absence or presence of oxygen. Pyruvate is the theoretical endpoint of glycolysis, but continued fermentative metabolism requires the reduction of pyruvate to lactate or the reduction of the pyruvate derivative acetaldehyde to ethanol, this latter process being the major fermentative outcome in yeast. In the presence of oxygen, pyruvate can alternatively be subjected to complete oxidation using enzymes of the tricarboxylic acid (TCA) cycle, with electrons stripped from pyruvate being ultimately donated to oxygen. Consequently, one outcome of glucose metabolism in yeast (and many organisms) is the coupling of glycolysis (production of pyruvate) to the oxidative degradation of pyruvate by the TCA cycle with the ultimate transfer of electrons gathered during these processes to oxygen (cellular respiration), resulting in the generation of ATP. However, if glucose is plentiful S. cerevisiae will metabolize glucose largely (but not exclusively) by fermentation as it provides the most rapid way to gain sufficient energy for biosynthesis and cell growth. As glucose becomes limiting, cellular respiration is engaged to extract energy from alternative carbon sources, principally the ethanol that was the end-product during the fermentative phase of growth.

Oxidation of pyruvate in yeast requires a functional mitochondrial electron transport chain. The passage of electrons through the electron transport chain is coupled to the establishment of a proton gradient across the inner mitochondrial membrane. This proton gradient is then used for a number of important mitochondrial processes. The most obvious use of the proton gradient is to power the synthesis of ATP via the mitochondrial ATP synthase (also known as the F₁Fₒ-ATPase). The transport of metabolites and proteins across the inner mitochondrial membrane is also dependent upon the membrane potential established by the proton gradient, and is in fact essential for cell viability. It is possible to completely inhibit mitochondrial ATP synthase and generate ATP by glycolysis and cells will remain viable. However, if the electrical gradient across the mitochondrial membrane is dissipated, cells will die because essential biochemical pathways housed in the mitochondrial matrix are no longer functional (see Pedersen, P. L., J Bioenerg Biomembr, 31(4): p. 291-304 (1999)). Importantly, if the inner mitochondrial membrane potential is too low, numerous process throughout the cell become compromised and in yeast the growth rate is significantly reduced (Veatch, J. R., et al., Cell, 137(7): p. 1247-58 (2009) and Francis, B. R., K. H. White, and P. E. Thorsness, J Bioenerg Biomembr, 39(2): p. 127-44, (2007)).

Non-respiring yeast (ρ° or ρ−) lack mitochondrial DNA and have been shown to have enhanced fermentative outcomes with respect to the yield of ethanol production (Toksoy et al. Applied and environmental microbiology, 71(10): p. 6443-5 (2005) and Dikicioglu, D., et al., Applied and environmental microbiology, 74(18): p. 5809-16 (2008)), presumably because available pyruvate is not lost to oxidation. Despite this, respiring yeast (ρ⁺) have been preferred for fermentation because they grow more robustly than ρ° yeast. Table 1 below shows a list of isolated mutations in mitochondrial ATP synthase genes that may be used for the dominant mitochondrial ATP synthase gene mutation of the present disclosure and discussed further in the construct of FIGS. 4a, 4b, FIG. 5 and FIGS. 6a, 6b and 6c. The ATP mutations enhance the ability of non-respiring yeast (ρ° or ρ−) yeast to grow, by 30%, so that ρ° growth rates approach those of wild-type respiring (ρ⁺) yeast.

TABLE 1
Dominant mutations in ATP1, ATP2, and ATP3 genes
that enhance F₁-ATPase activity, the membrane
potential of mitochondria, and growth of ρ° yeast.

| Allele | Amino Acid Location | Change |
|---|---|---|
| ATP1-75 | 102 | Asn to Ile |
| ATP1-111 | 111 | Val to Phe |
| ATP2-227 | 227 | Gly to Ser |
| ATP3-1 | 303 | Ile to Thr |
| ATP3-5 | 297 | Thr to Ala |

The dominant mitochondrial ATP synthase gene mutations listed in Table 1 improve the growth of non-respiring yeast in laboratory strains also lead to robust growth of ethanologenic strains currently used in commercial fermentative processes. Introduction of the ATP1, ATP2 or ATP3 mutations of Table 1 into ethanologenic non-respiring yeast strains that have been optimized for tolerance to environmental inhibitors and modified to ferment a diversity of saccharides will significantly enhance fermentation and ethanol production. These dominant mutations encode the alpha, beta and gamma subunits of the F₁ subunit of mitochondrial ATP synthase. The molecular basis by which the mutation optimizes the growth of ρ° yeast is via an enhancement of mitochondrial membrane potential due to increased hydrolysis of ATP and the consequent enhanced flux of ATP/ADP exchange across the inner mitochondrial membrane. The mutation optimizes ρ° yeast growth and thus increases the efficiency of fermentation in industrial strains of yeast by allowing them to grow robustly as ρ° derivatives. Additionally, the use of the methods described in FIG. 1, FIG. 2 and FIG. 3 provides additional approaches for enhancing the fermentation capability of industrial strains of yeast.

The advantages of using vigorous non-respiring yeast (ρ° or ρ−) in fermentations includes allowing for less rigorous fermentation conditions, such as an anaerobic environment not being required for the yeast to efficiently ferment and not undertake respiratory metabolism, as well as producing a greater yield of ethanol per unit of glucose metabolized (no oxidative metabolism of ethanol as glucose becomes limiting). Hence, the introduction of the ATP1-111 mutation (SEQ ID NO:1 or SEQ ID NO:2) or other dominant mutant alleles of ATP1, ATP2, or ATP3 that enhances the fermentative growth of non-respiring yeast into ethanologenic yeast strains that can be used in commercial applications will be a significant technical advance. Importantly, as will be discussed further in the Examples listed below, these dominant mutations grow as robustly as the parental yeast strain bearing intact mitochondrial DNA and produce as much as 25% more ethanol than the ρ⁺ strain when given the same amount of glucose with doubling times equal to the ρ⁺ strain.

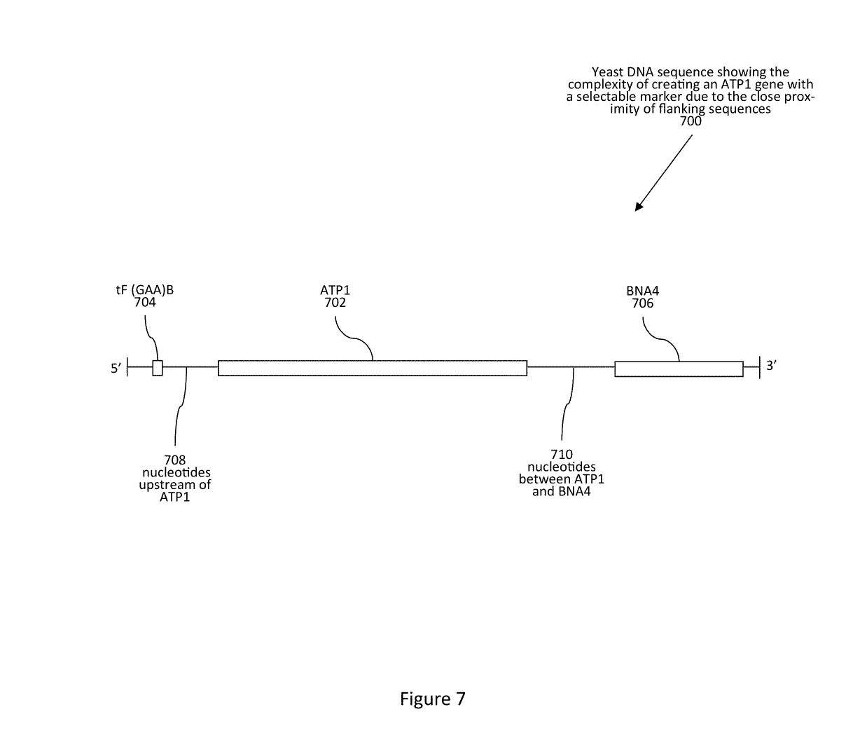

As shown in FIG. 4a and FIG. 4b, a flow diagram for the construction of a DNA construct for the expression of a dominant mitochondrial ATP synthase gene mutation, such as the ATP1-111 allele 400 is provided. As discussed in FIG. 7, due to the complex nature and limited space available in the regions flanking the dominant mitochondrial ATP synthase gene mutation in yeast the construction and introduction of a DNA construct comprising the dominant mitochondrial ATP synthase gene mutation and a selectable marker (the KAN-MX gene) required precise placement. In FIG. 4a, starting at the 5′ ATP1 UTR 402 a dominant mitochondrial ATP synthase gene mutation is shown, such as the ATP1-111 mutation 404 (SEQ ID NO:1 or SEQ ID NO:2). On the 3′ ATP UTR 406 end a selectable marker 408 such as the KAN-MX resistance marker is shown, where the selectable marker 408 and the dominant mitochondrial ATP synthase gene mutation 404 are operably linked. A repetitive sequence (SEQ ID NO:3) comprising 200 base pairs of DNA sequence is immediately 5′ to the ATP1-111 404 open reading frame and a second repetitive sequence (SEQ ID NO:4) comprising the 200 base pairs of DNA sequence is immediately 3′ to the ATP1-111 404 ORF. These repetitive sequences (SEQ ID NO:3 and SEQ ID NO:4) have proven recalcitrant to molecular cloning and historically created confusion as to the genomic structure in this region of chromosome I. A second set of directly repeated sequences (SEQ ID NO:5) are located immediately 5′ and 3′ to the selectable marker 408. As shown in FIG. 4b, each of the components of FIG. 4a is operably linked to the next, i.e., starting at the 5′ ATP1 UTR 402, the ATP1-111 mutation 404 is operably linked 410 to the KAN-MX selectable marker 408 on the 3′ ATP UTR 406. The construct 400 is then integrated into a yeast and yeast expressing the ATP1-111 allele are then generated. All DNA manipulations were performed using standard techniques (see Sambrook, et al., Molecular Cloning, 2nd Ed., Cold Spring Harbor Laboratory Press, Cold Spring Harbor, N.Y. (1989)). Restriction and DNA modification enzymes were purchased from New England Biolabs unless otherwise noted. Plasmid DNA was prepared from E. coli by boiling lysis (Sambrook et al., (1989)).

The ATP1-111 mutation is a suppressor of the slow-growth non-respiring yeast lacking mitochondrial DNA due to the substitution of phenylalanine for valine at position 111 of the alpha-subunit of mitochondrial ATP synthase (Atp1p in yeast). The suppressing activity of ATP1-111 requires intact beta (Atp2p) and gamma (Atp3p) subunits of mitochondrial ATP synthase, but not the stator stalk subunits b (Atp4p) and OSCP (Atp1p). ATP1-111 and other similarly suppressing mutations in ATP1 and ATP3 increase the growth rate of wild-type strains lacking mitochondrial DNA. These suppressing mutations decrease the growth rate of yeast containing an intact mitochondrial chromosome on media requiring oxidative phosphorylation, but not when grown on fermentable media.

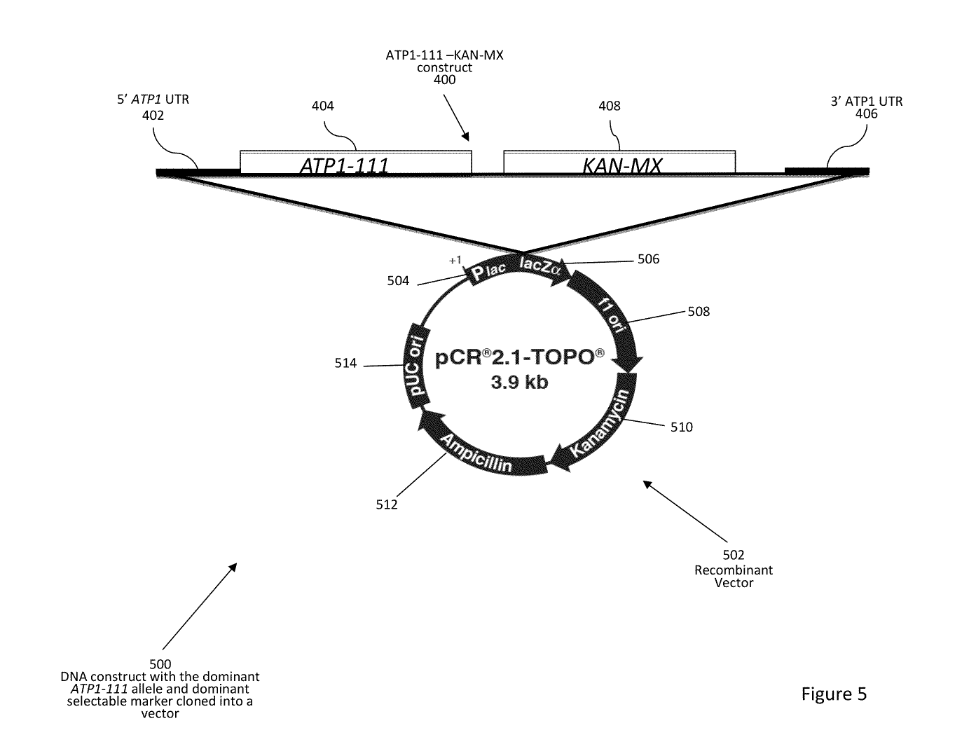

FIG. 5 shows the integration of the ATP1-111 construct 400 of FIGS. 4a and 4b into a recombinant vector 500. As shown in FIG. 5, starting at the 5′ ATP1 UTR 402 of the ATP1-1,1-KAN-MX construct 400, the ATP1-111 dominant mutation 404 and the KAN-MX selectable marker 408 is cloned into a standard E. coli recombinant DNA vector 502 (a pCR 2.1-TOPO 3.9 kb) using standard genetic techniques. The recombinant vector 502 comprises a promoter (Plac) 504 which is operably linked to a lacZ gene 506. The lacZ gene 506 expresses an intracellular enzyme that cleaves disaccharide lactose into glucose and galactose. The lacZ gene 506 is operably linked to the f1 origin of replication sequence (f1 ori) 508. The f1 ori 508 sequence is operably linked to a kanamycin resistance gene (kanamycin) 510. The kanamycin resistance gene 510 is used in the vector 502 as a selectable marker where the yeast expressing the kanamycin resistance gene 510 are able to express resistance to a kanamycin antibiotic when grown in media containing the kanamycin antibiotic. The kanamycin resistance gene 510 is operably linked to an ampicillin resistance gene (ampicillin) 512 where the ampicillin resistance gene 512 is also used as a selectable marker in a similar manner as that of the kanamycin resistance gene 510. The ampicillin resistance gene 512 is operably linked to the pUC origin of replication (pUC ori) 514.

As shown in FIG. 6a, FIG. 6b and FIG. 6c, a flow diagram of the introduction of the dominant mitochondrial ATP synthase gene mutation into a recipient yeast strain and then the removal of a selectable marker from the ATP1-1,1-KAN-MX construct that has been integrated into the chromosome is provided 600. In FIG. 6a, the construct of FIG. 4b is shown comprising, starting at the 5′ ATP UTR 402, the ATP1-111 allele 404 operably linked to the KAN-MX selectable marker 408 where the KAN-MX selectable marker 408 is flanked by directly repeated sequences (SEQ ID NO:5) 602 408 on the 3′ ATP UTR 406. In FIG. 6b, the directly repeated sequences (SEQ ID NO:5) 602 undergo frequent spontaneous recombination, leading to loss of the KAN-MX sequence 408 and retention of ATP1-111 and one repeated sequence 404. These 200 nucleotides of the repeated sequences (SEQ ID NO:5) are derived from the hisG gene of E. coli and bear no significant similarity to S. cerevisiae sequence. One copy of this repeated sequence 602 or functionally similar sequence is placed immediately 5′ to the gene encoding the dominant drug marker, such as KAN-MX. A second copy of the repeated sequence 602 or a functionally similar sequence is placed immediately 3′ to the gene encoding the dominant drug marker, such as KAN-MX. Homologous recombination in yeast will frequently lead to loss of the sequence 408 located between the direct repeats 602. Such spontaneous losses can be found by identifying yeast colonies that lack the dominant drug marker, such as yeast formerly resistant to kanamycin-like drugs will now be sensitive to the drug. Hence, yeast colonies will be replica plated from media lacking the drug to media containing the drug. Those colonies unable to grow in the presence of the drug will be recovered from the drug-free media and the genetic structure verified by PCR. Kanamycin sensitive isolates will still contain the ATP1-111 mutation 404. FIG. 6c shows the ATP1-111 construct 400 with the ATP1-111 mutation 404 but without the KAN-MX selectable marker. This allows the conversion of the recipient industrial yeast strain with all of its positive attributes of ethanol, heat, and acid tolerance intact, to a non-respiring strain (lacking mitochondrial DNA) that grows at the same robust rate but has the significant advantage of increased ethanol yield (approximately 25%) from a given unit of input sugar.

FIG. 7 provides a diagram of a yeast DNA sequence showing the complexity of creating an ATP1 gene construct with a selectable marker due to the close proximity of flanking sequences 700. As shown in FIG. 7, the gene labeled “tF(GAA)B” 704 encodes a phenylalanine tRNA and resides only 565 nucleotides upstream 708 of the ATP1 702 start codon. As tRNA genes have poorly defined promoter elements located 3′ to the gene, the region for manipulation of the ATP1 locus in the 5′ region of ATP1 702 is therefore small and poorly defined. The gene located 3′ to ATP1 702 is the BNA4 gene 706 which encodes a protein necessary for the synthesis of NAD+. There are 455 nucleotides 710 between the ATP1 702 stop codon and the start codon of BNA4 gene 706. In order to incorporate the repetitive sequence discussed in FIG. 4b associated with the ATP1 gene, the repetitive sequences of SEQ ID NO:4 in the 3′ region 710 of ATP1 702 needs to avoid 3′ expression elements of the ATP1 gene 702 along with the 5′ promoter elements of the BNA4 gene 706. The region between the ATP1 gene 702 and the BNA4 gene 706 creates a small intragenic region where changes can be made, including introducing the operably linked KAN-MX genetic tag into this region. Adding to this challenge, published reports have presented evidence for three tandem linked copies of ATP1 on chromosome II of multiple characterized yeast strains, including those used in generating the yeast genome sequence (Takeda et. al., Yeast, 15, 873-878 (1999)). Despite these complications, as shown in FIGS. 4a, 4b, FIG. 5 and FIGS. 6a, 6b and 6c, the dominant mitochondrial ATP synthase gene mutation has been successfully tagged and cloned.

As used herein “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

A variety of transformation techniques are available and known to those skilled in the art for introduction of constructs into a yeast. As described earlier, all DNA manipulations were performed using standard techniques (Sambrook et al., (1989)). To confirm the presence of the transgenes or the absence of genes in yeast, including the COX1, COX2, COX3, or COB gene or CAT5 gene, a polymerase chain reaction (PCR) amplification or Southern blot analysis can be performed using methods known to those skilled in the art.

Generally, the DNA that is introduced into an organism is part of a construct, as described in FIG. 4b. A construct is an artificially constructed segment of DNA that may be introduced into a target organism tissue or organism cell. Constructs are engineered DNA molecules that encode genes and flanking sequences that enable the constructs to integrate into the host genome at (targeted) locations. The DNA may be a gene of interest, e.g., a coding sequence for a protein, or it may be a sequence that is capable of regulating expression of a gene, such as an antisense sequence, a sense suppression sequence, or a miRNA sequence. As used herein, “gene” refers to a segment of nucleic acid. A gene can be introduced into a genome of a species, whether from a different species or from the same species. The construct typically includes regulatory regions operably linked to the 5′ side of the DNA of interest and/or to the 3′ side of the DNA of interest. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation. A cassette containing all of these elements is also referred to herein as an expression cassette. The expression cassettes may additionally contain 5′ leader sequences in the expression cassette construct. (A leader sequence is a nucleic acid sequence containing a promoter as well as the upstream region of a gene.) The regulatory regions (i.e., promoters, transcriptional regulatory regions, translational regulatory regions, and translational termination regions) and/or the polynucleotide encoding a signal anchor may be native/analogous to the host cell or to each other. Alternatively, the regulatory regions and/or the polynucleotide encoding a signal anchor may be heterologous to the host cell or to each other. See, U.S. Pat. No. 7,205,453 and U.S. Patent Application Publication Nos. 2006/0218670 and 2006/0248616. The expression cassette may additionally contain selectable marker genes which will be discussed in more detail later.

The products of the genes are often proteins, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process may be modulated, including the transcription, up-regulation, RNA splicing, translation, and post translational modification of a protein.

A promoter is a DNA region, which includes sequences sufficient to cause transcription of an associated (downstream) sequence. The promoter may be regulated, i.e., not constitutively acting to cause transcription of the associated sequence. If inducible, there are sequences present therein which mediate regulation of expression so that the associated sequence is transcribed only when an inducer molecule is present. The promoter may be any DNA sequence that shows transcriptional activity in the chosen yeast cell. The promoter may be inducible or constitutive. It may be naturally-occurring, may be composed of portions of various naturally-occurring promoters, or may be partially or totally synthetic. Guidance for the design of promoters is derived from studies of promoter structure, such as that of Harley and Reynolds, Nucleic Acids Res., 15, 2343-61 (1987). In addition, the location of the promoter relative to the transcription start may be optimized. Many suitable promoters for use in yeast are well known in the art, as are nucleotide sequences, which enhance expression of an associated expressible sequence.

While the lac promoter is an example of a promoter that may be used, a number of promoters may be used herein. Promoters can be selected based on the desired outcome. That is, the nucleic acids can be combined with constitutive, tissue-preferred, or other promoters for expression in the host cell of interest. The promoter may be inducible or constitutive. It may be naturally-occurring, may be composed of portions of various naturally-occurring promoters, or may be partially or totally synthetic. In addition, the location of the promoter relative to the transcription start may be optimized. Many suitable promoters for use in yeast are well known in the art, as are nucleotide sequences, which enhance expression of an associated expressible sequence.

### COX1, COX2, COX3 and COB Genes

Subunit I (CoxI) of cytochrome c oxidase is a protein subunit of the terminal member of the mitochondrial inner membrane electron transport chain. The gene that encodes this protein is encoded on mitochondrial DNA and is designated COX1.

Cytochrome c oxidase subunit II, abbreviated COX2, is the second subunit of cytochrome c oxidase. Cytochrome c oxidase is an oligomeric enzymatic complex which is a component of the respiratory chain and is involved in the transfer of electrons from cytochrome c to oxygen. In eukaryotes this enzyme complex is located in the mitochondrial inner membrane; in aerobic prokaryotes it is found in the plasma membrane.

Subunit II (Cox2) transfers the electrons from cytochrome c to the catalytic subunit 1. It contains two adjacent transmembrane regions in its N-terminus and the major part of the protein is exposed to the periplasmic or to the mitochondrial intermembrane space, respectively. Cox2 provides the substrate-binding site and contains a copper centre called Cu(A), probably the primary acceptor in cytochrome c oxidase. An exception is the corresponding subunit of the cbb3-type oxidase which lacks the copper A redox-centre.

Subunit III (Coxa) of cytochrome c oxidase, the gene encoding this protein is abbreviated COX3, is the terminal member of the mitochondrial inner membrane electron transport chain. Cytochrome c oxidase is an oligomeric enzymatic complex which is a component of the respiratory chain and is involved in the transfer of electrons from cytochrome c to oxygen. In eukaryotes this enzyme complex is located in the mitochondrial inner membrane; in aerobic prokaryotes it is found in the plasma membrane.

Cytochrome b, abbreviated Cobp, is the mitochondrially encoded subunit of the ubiquinol-cytochrome c reductase complex. This multisubunit protein complex, also known as complex III, is located in the mitochondrial inner membrane. The gene on the mitochondrial chromosome that encodes this protein is named COB.

### CAT5

CAT5 is a ubiquinone biosynthesis gene found in yeast. The deletion of the CAT5 gene decreases respiratory growth by precluding electron transport from contributing to membrane potential in mitochondria, but unlike the loss of mtDNA, the formation of coupled F1 Fo-ATPase is not impaired.

### Yeast Strain Production

Standard genetic techniques were used to construct and analyze the various strains of the present disclosure (see Sherman et al., 1986). Escherichia coli strain XL-1 Blue (Stratagene) was used for preparation and manipulation of DNA. Plasmids containing E. coli were grown in Luria-Bertani (LB) broth supplemented with 125 μg/ml ampicillin (Sambrook et al., (1989)). Yeast strains were grown in rich glucose medium (YPD) containing 2% glucose, 2% Bacto peptone, 1% yeast extract (Difco), 40 mg/l adenine and 40 mg/l tryptophan; rich ethanol glycerol medium (YPEG) containing 3% ethanol, 3% glycerol, 2% Bacto peptone, 1% yeast extract (Difco), 40 mg/l adenine and 40 mg/l tryptophan; rich raffinose medium (YPR) in which filter sterilized raffinose replaced glucose in the YPD formulation; synthetic glucose medium (SD) containing 2% glucose, 6.7 g/l Yeast Nitrogen Base without amino acids (Difco) supplemented with appropriate nutrients; synthetic ethanol glycerol medium (SEG) containing 3% ethanol, 3% glycerol, 6.7 g/l Yeast Nitrogen Base without amino acids (Difco) supplemented with appropriate nutrients; and sporulation medium (SPO) containing 1% potassium acetate supplemented with the complete set of nutrients. The complete set of nutrients is uracil 40 mg/l, adenine 40 mg/l, tryptophan 40 mg/l, lysine 60 mg/l, leucine 100 mg/l, histidine 20 mg/l, isoleucine 30 mg/l, and valine 150 mg/l. For plates, bacteriological agar (US Biological) was added at 15 g/l. Where indicated, ethidium bromide (EtBr) was added at 25 μg/ml and geneticin at 300 μg/ml, or nourseothricin (Werner Bioagents) was top spread on plates at 25 μg/ml. All yeast media were incubated at 30.0 except SPO, which was incubated at room temperature. LB medium was incubated at 37° C. When ρ° strains were specifically used (FIGS. 4B, 7, 8 and 9; Table 2), the corresponding ρ+ strain was converted to ρ° by serial culturing in SD liquid media containing 25 μg/ml EtBr (Fox et al., 1991).

### ATP1 Sequencing

PCR primers for ATP1 sequencing were (SEQ ID NO:6) (forward) (SEQ ID NO:7) (reverse). For cloning, ATP1 alleles were PCR amplified from genomic DNA using Pfu Turbo DNA polymerase (Stratagene). Primers were (SEQ ID NO:8) (forward) and (SEQ ID NO:9) (reverse).

### Mitochondrial Isolation

Isolation of mitochondria, immuno-detection of proteins and measurement of F1Fo-ATPase activity Mitochondrial isolation was performed as described by Yaffe, 1991. Cells were grown in 1 liter of YPR to OD600=1.5. Mitochondrial yield was determined using the Coomassie Protein Assay (Pierce). ATPase activities were determined using isolated mitochondria essentially as described (see Tzagoloff, Methods Enzymol 55:351-358, (1979)). Reaction mixtures contained 120 μg of mitochondria and were incubated at 37.0 for 12 minutes.

### Vector Construction, Transformation, and Heterologous Protein Expression

As used herein plasmid, vector or cassette refers to an extrachromosomal element often carrying genes and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with an appropriate 3′ untranslated sequence into a cell.

While one example of an expression vector is the recombinant vector of FIG. 5, derivatives of the vectors described herein may be capable of stable transformation of many yeast cells. Vectors for stable transformation of yeast are well known in the art and can be obtained from commercial vendors or constructed from publicly available sequence information. Expression vectors can be engineered to produce heterologous and/or homologous protein(s) of interest (e.g., antibodies, mating type agglutinins, etc.). Such vectors are useful for recombinantly producing the protein of interest and for modifying the natural phenotype of host cells.

To construct the vector, the upstream DNA sequences of a gene expressed under control of a suitable promoter may be restriction mapped and areas important for the expression of the protein characterized. The exact location of the start codon of the gene is determined and, making use of this information and the restriction map, a vector may be designed for expression of a heterologous protein by removing the region responsible for encoding the gene's protein but leaving the upstream region found to contain the genetic material responsible for control of the gene's expression. A synthetic oligonucleotide is inserted in the location where the protein sequence once was, such that any additional gene could be cloned in using restriction endonuclease sites in the synthetic oligonucleotide (i.e., a multicloning site). Publicly available restriction proteins may be used for the development of the constructs. An unrelated gene (or coding sequence) inserted at this site would then be under the control of an extant start codon and upstream regulatory region that will drive expression of the foreign (i.e., not normally present) protein encoded by this gene. Once the gene for the foreign protein is put into a cloning vector, it can be introduced into the host organism using any of several methods, some of which might be particular to the host organism. Variations on these methods are described in the general literature. Manipulation of conditions to optimize transformation for a particular host is within the skill of the art.

The basic techniques used for transformation and expression in yeast are known in the art. Exemplary methods have been described in a number of texts for standard molecular biological manipulation (see Sambrook et al. (1989)). These methods include, for example, biolistic devices (See, for example, Sanford, Trends In Biotech., 6: 299-302, (1988)); U.S. Pat. No. 4,945,050; use of a laser beam, electroporation, microinjection or any other method capable of introducing DNA into a host cell (e.g., an NVPO).

To confirm the presence of the transgenes in transgenic cells, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. Once transgenic organisms have been obtained, they may be grown to produce organisms or parts having the desired phenotype.

### Selectable Markers

A selectable marker (SM) such as the KAN-MX gene of the construct of FIG. 4b, can provide a means to identify yeast cells that express a desired product. Selectable markers include, but are not limited to, ampicillin resistance for prokaryotes such as E. coli, neomycin phosphotransferase, which confers resistance to the aminoglycosides neomycin, kanamycin and paromycin (Herrera-Estrella, EMBO J. 2:987-995, (1983)); dihydrofolate reductase, which confers resistance to methotrexate (Reiss, Plant Physiol. (Life Sci. Adv.) 13:143-149, (1994)); trpB, which allows cells to utilize indole in place of tryptophan; hisD, which allows cells to utilize histinol in place of histidine (Hartman, Proc. Natl. Acad. Sci., USA 85:8047, (1988)); mannose-6-phosphate isomerase which allows cells to utilize mannose (WO 94/20627); hygro, which confers resistance to hygromycin (Marsh, Gene 32:481-485, (1984)); ornithine decarboxylase, which confers resistance to the ornithine decarboxylase inhibitor, 2-(difluoromethyl)-DL-ornithine (DFMO; McConlogue, In: Current Communications in Molecular Biology, Cold Spring Harbor Laboratory ed., (1987)); deaminase from Aspergillus terreus, which confers resistance to Blasticidin S (Tamura, Biosci. Biotechnol. Biochem. 59:2336-2338, (1995)); phosphinothricin acetyltransferase gene, which confers resistance to phosphinothricin (White et al., Nucl. Acids Res. 18:1062, (1990); Spencer et al., Theor. Appl. Genet. 79:625-633, (1990)); a mutant acetolactate synthase, which confers imidazolione or sulfonylurea resistance (Lee et al., EMBO J. 7:1241-1248, (1988)), a mutant EPSPV-synthase, which confers glyphosate resistance (Hinchee et al., BioTechnology 91:915-922, (1998)); a mutant psbA, which confers resistance to atrazine (Smeda et al., Plant Physiol. 103:911-917, (1993)), a mutant protoporphyrinogen oxidase (see U.S. Pat. No. 5,767,373), or other markers conferring resistance to an herbicide such as glufosinate.

### Transcription Terminator

The transcription termination region of the constructs is a downstream regulatory region including the stop codon and the transcription terminator sequence. Alternative transcription termination regions that may be used may be native with the transcriptional initiation region, may be native with the DNA sequence of interest, or may be derived from another source. The transcription termination region may be naturally occurring, or wholly or partially synthetic.

The practice described herein employs, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, et al., Molecular Cloning, Cold Spring Harbor Laboratory Press, Cold Spring Harbor, N.Y. (1982); Sambrook, et al., (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, NY (1992); Guthrie and Fink, Guide to Yeast Genetics and Molecular Biology, Academic Press, NY (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire, et al., RNA Interference Technology From Basic Science to Drug Development, Cambridge University Press, Cambridge (2005); Schepers, RNA Interference in Practice, Wiley-VCH (2005); Engelke, RNA Interference (RNAi): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004)).

### EXAMPLES

The following examples are provided to illustrate further the various applications and are not intended to limit the invention beyond the limitations set forth in the appended claims.

### Example 1

### Doubling Times of Yeast Strains in Rich Glucose Media (YPD)

Table 2 below shows the growth doubling time of related yeast strains, differing from each other on the basis of whether or not they contain mitochondrial DNA and consequently a functional electron transport chain. Column 1 shows the yeast strain name, column 2 shows the relevant yeast genotype and column 3 shows the doubling time of the yeast strain grown in a YPD media. As shown in Table 2, mutations in mitochondrial ATP synthase genes have been provided that enhance the ability of non-respiring yeast (ρ°) yeast to grow by 30%, so that ρ° growth rates approach those of wild-type ρ⁺ yeast, see in particular strains PTY44 and BFY111. As also shown in Table 2, transgenic non-respiring yeast bearing mitochondrial genome lacking the COX3 gene showed a doubling time also approaching the doubling times of wild-type ρ⁺ yeast.

TABLE 2
Doubling times of yeast strains with indicated genotypes in rich
glucose media (YPD). Doubling times are in units of hours

| Strain Name | Relevant Genotype | Doubling Time |
|---|---|---|
| PTY44 | Wild Type | 1.5 ± 0.1 |
| BFY141 | ATP1-111 | 1.5 ± 0.1 |
| KWY94 | ATP1-75 | 1.7 ± 0.1 |
| KWY96 | ATP3-1 | 1.6 ± 0.1 |
| PTY100 | ATP3-5 | 1.6 ± 0.1 |
| PTY52 | yme1 | 1.8 ± 0.1 |
| BFY138 | yme1ATP1-111 | 1.8 ± 0.1 |
| PTY93 | yme1ATP1-75 | 1.7 ± 0.1 |
| PTY109 | yme1ATP3-1 | 1.7 ± 0.1 |
| KWY91 | yme1ATP3-5 | 1.8 ± 0.1 |
| TCY1 | cat5 | 2.1 ± 0.1 |
| KWY116 | cat5 ATP1-111 | 12.0 ± 0.1 |
| PTY44 ρ° | Wild Type ρ° | 2.5 ± 0.1 |
| BFY141 ρ° | ATP1-111 ρ° | 1.7 ± 0.1 |
| KWY94 ρ° | ATP1-75 ρ° | 2.0 ± 0.1 |
| KWY96 ρ° | ATP3-1 ρ° | 1.9 ± 0.1 |
| PTY100 ρ° | ATP3-5 ρ° | 2.0 ± 0.1 |
| BFY138 ρ° | yme1ATP1-111 ρ° | 2.4 ± 0.1 |
| PTY93 ρ° | yme1ATP1-75 ρ° | 3.2 ± 0.1 |
| PTY109 ρ° | yme1ATP3-1 ρ° | 3.4 ± 0.1 |
| KWY91 ρ° | yme1ATP3-5 ρ° | 3.3 ± 0.1 |
| TCY1 ρ° | cat5 ρ° | 2.8 ± 0.1 |
| KWY116 ρ° | cat5 ATP1-111 ρ° | 2.1 ± 0.1 |
| JSC350X | cox3-5 | 1.7 ± 0.1 |

### Example 2

### ATPase Mutation Activity in Yeast

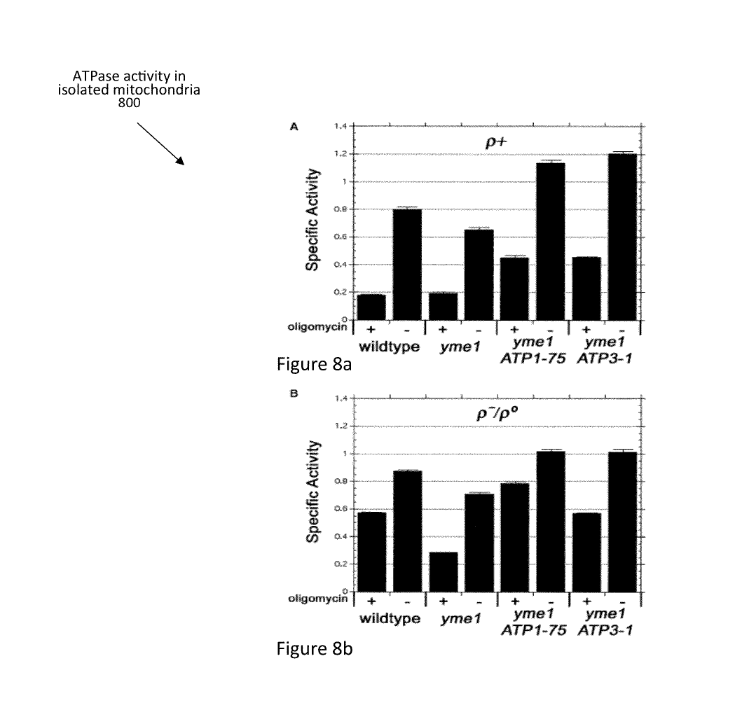

FIG. 8a and FIG. 8b shows F₁-ATPase activity in yeast strains bearing dominant mitochondrial ATP synthase gene mutations, with and without mitochondrial DNA 800. As shown in FIG. 8a and FIG. 8b, five micrograms of ρ+ (FIG. 8a) or ρ° (FIG. 8b) mitochondria isolated from a wild-type yeast, a yme1 yeast, a yme1 ATP1-75 yeast and a yme1 ATP3-1 yeast were assayed in triplicate. Data are shown in means+/−standard error of the mean. Reactions were incubated without (−) or with (+) oligomycin (2 μg/ml) to determine the fraction of inhibited ATPase activity. ATPase specific activity is expressed as [micromoles of Pi per minute per microgram of protein (×1000)]. FIG. 8a and FIG. 8b shows that the molecular basis by which these mutations (ATP1-75 and ATP3-1) optimize the growth of ρ° yeast via an enhancement of mitochondrial membrane potential due to increased hydrolysis of ATP.

### Example 3

### Generation of an Inner Mitochondrial Membrane Potential in ρ+ and ρ° Yeast by Addition of ATP

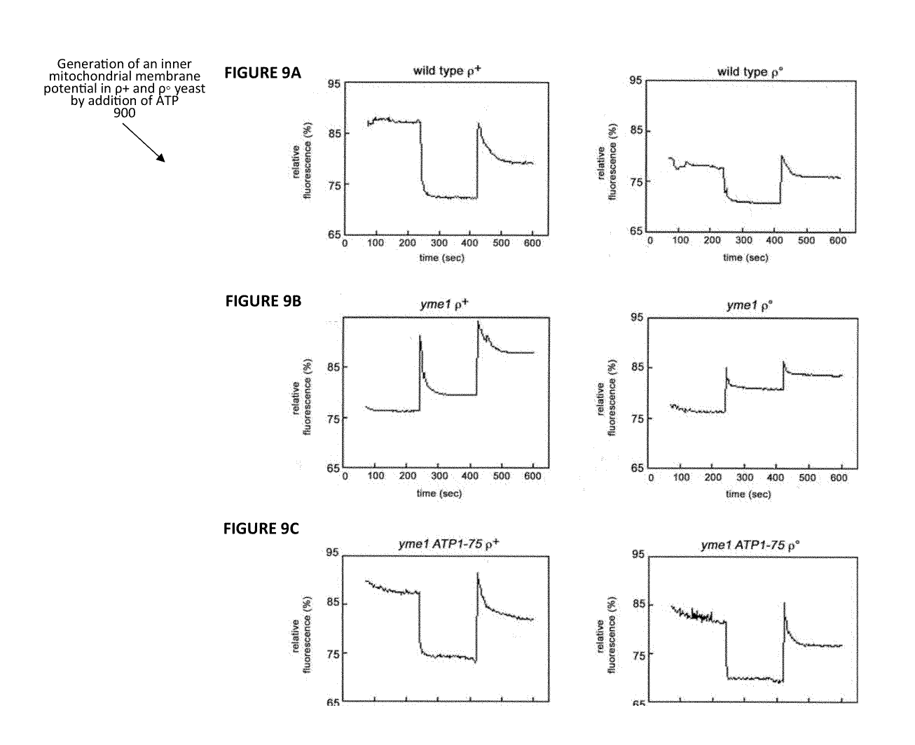

FIGS. 9A, 9B and 9C provides six graphs showing examples of the generation of an inner mitochondrial membrane potential in wild-type, yme1, and yme1 ATP-75 yeast. Mitochondria were isolated from wild-type, yme1, and yme1 ATP-75 yeast. The mitochondria prepared from wild-type of FIG. 9A, and yme1 of FIG. 9B and ATP 1-75 strains of FIG. 9C essentially as described by Yaffe, Methods Enzymol.; Vol. 194, pp 627-643 (1991). The mitochondria prepared from the yme1 strain were generated from a batch culture of ρ⁺ cells by treatment with ethidium bromide. The potential dependent quenching of rhodamine 123 fluorescence is expressed as percentage of relative fluorescence. ATP was added at 240 sec, and the ionophore valinomycin was added at 420 sec. As shown in FIG. 9A, FIG. 9B and FIG. 9C, the enhanced flux of ATP/ADP exchange across the inner mitochondrial membrane allows the ATP1-75 mutation to optimize ρ° yeast growth and thus increase the efficiency of fermentation in industrial strains of yeast by allowing them to grow robustly as ρ° derivatives.

### Example 4

### Ethanol Production in Yeast During Batch Fermentation

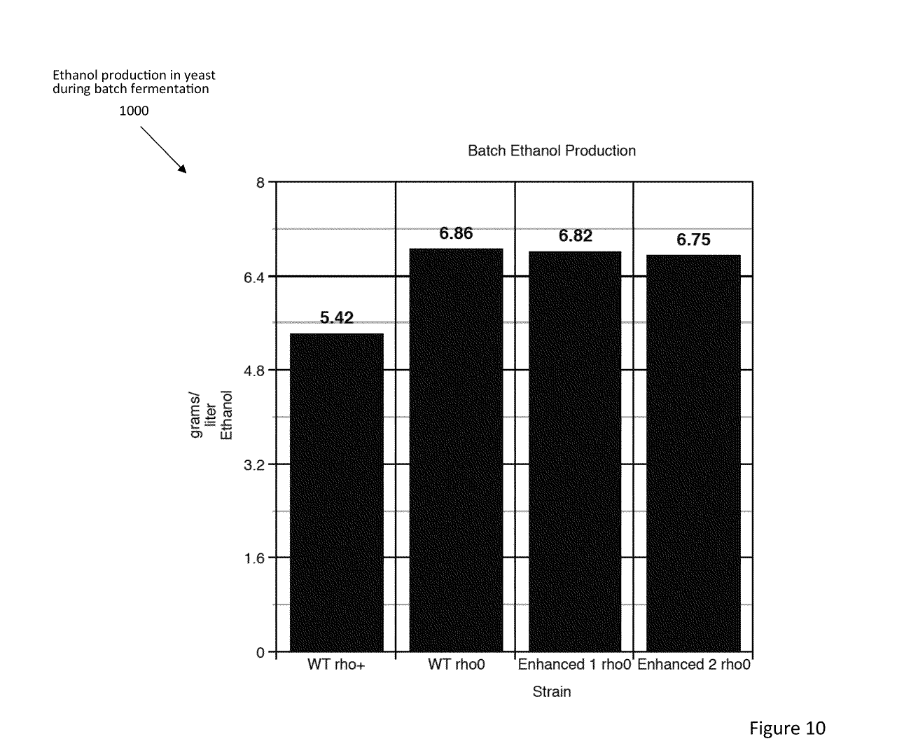

FIG. 10 provides a graph showing enhanced ethanol production in non-respiring (p°) yeast of the present disclosure, including enhanced yeast strains bearing the ATP 1-111 mutation as well as the ATP1-75 mutation. As shown in FIG. 10, four yeast are provided. The first yeast, a wild type (WT) ρ+ yeast produced 5.42 g/liter of ethanol. The second yeast, a non-respiring WT ρ° yeast, produced 6.86 g/liter of ethanol. The third yeast, a non-respiring ρ° strain with the ATP1-111 mutation produced 6.82 g/liter and the fourth yeast, and the fourth yeast was a non-respiring ρ° strain with the ATP1-75 mutation produced 6.75 g/liter of ethanol. Yeast were incubated in rich glucose media until glucose was exhausted and ethanol yields measured using alcohol dehydrogenase (NADH formation). The ρ° strain with the ATP1-111 mutation and the ρ° strain with the ATP1-75 mutation grow as robustly as the parental yeast strain (Table 2) bearing intact mitochondrial DNA (referred to as ρ⁺ yeast) and produce as much as 25% more ethanol than the ρ⁺ strain when given the same amount of glucose.

### Example 5

### Ethanol Production in Yeast Bearing a Mitochondrial Genome Lacking the COX3 Gene During Batch Fermentation

Table 3 below provides comparative growth and ethanol production in batch fermentation of yeast bearing intact mitochondrial DNA (Rho+), yeast lacking mitochondrial DNA (Rho0), yeast lacking mitochondrial DNA but containing the ATP1-111 mutation (ATP1-111 Rho0), and yeast bearing a mitochondrial genome lacking the COX3 gene (Rho+Mit−).

TABLE 3

| | | | ATP1-111 | |
|---|---|---|---|---|
| Strain | Rho+ | Rho0 | Rho0 | Rho+ Mit− |
| Doubling Time | 2 ± 0.19 | 2.73 ± 0.25 | 2.01 ± 0.28 | 1.72 ± 0.14 |
| (hours) | | | | |
| Ethanol | 5.36 ± 0.36 | 8.14 ± 1.9 | 8.24 ± 2.36 | 7.58 ± 0.04 |
| Concentration | | | | |
| at Stationary | | | | |
| (g/L) | | | | |

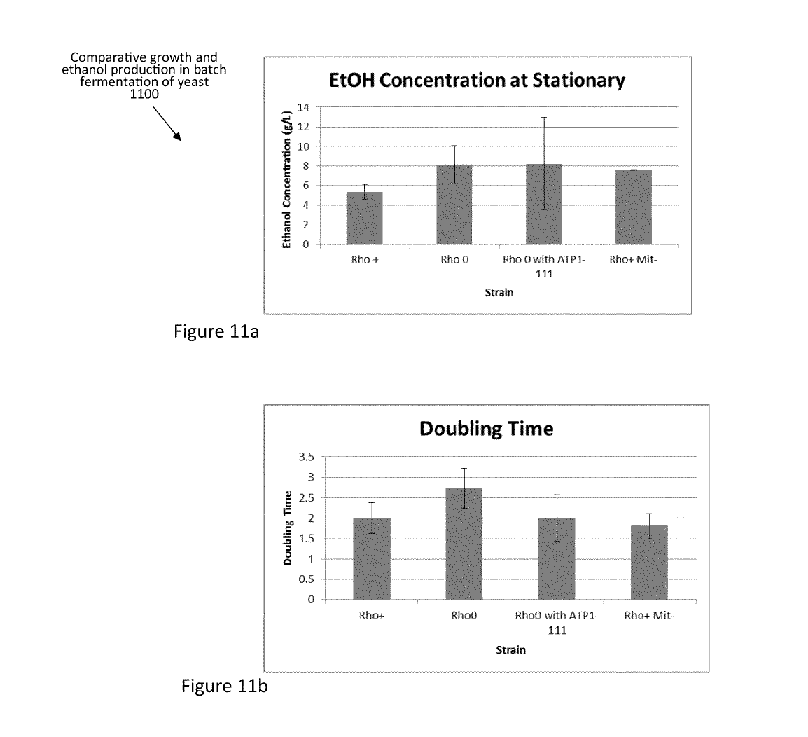

As shown in Table 3 and previously shown in FIG. 10, yeast containing intact mitochondrial genomes (Rho+) grow faster but produce less ethanol than yeast lacking mitochondrial DNA. Also shown in Table 3, with the data plots in the graphs shown in FIGS. 11a and 11b is evidence that yeast bearing a mitochondrial genome lacking the COX3 gene (or similarly COX 1, COX2, or COB) also produce more ethanol than yeast bearing intact mitochondrial genomes (7.58 g/l versus 5.36 g/l). Moreover, Table 3 and plotted in FIGS. 11a and 11b experimental data indicates that yeast lacking the COX3 gene grows (shown in doubling time by hours) just as robustly in rich glucose media as do strains containing intact mitochondrial DNA or strains lacking mitochondrial DNA entirely but also bearing the ATP1-111 mutation. In this example the mitochondrial genome lacking the COX3 gene has been transferred into an industrial diploid yeast strain and similarly enhances the production of ethanol in comparison to the standard yeast strain that contains intact mitochondria DNA (between 5% and 25% enhanced production) while still allowing allows rapid growth in glucose-rich conditions.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions, and sub-combinations as are within their true spirit and scope.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

What is claimed is:

1. A method for enhanced fermentation of plant material through the genetic modification of non-respiring yeast comprising:
removing a mitochondrial genome from a diploid yeast;
converting the diploid yeast to a spheroplast;
converting a kar1-1 yeast strain bearing a mitochondrial genome lacking a mitochondrial gene to a spheroplast;
fusing the diploid yeast to the kar1-1 yeast strain bearing the mitochondrial genome lacking a mitochondrial gene; and
producing a transgenic non-respiring yeast, wherein the non-respiring yeast is capable of fermenting plant material.

2. The method of claim 1, where the mitochondrial genome of said transgenic non-respiring yeast is lacking a mitochondrial gene chosen from the group comprising COX1, COX2, COX3, and COB.

3. The method of claim 2, further comprising introducing a dominant mitochondrial ATP synthase gene mutation into said yeast.

4. The method of claim 3, wherein the dominant mitochondrial ATP synthase gene mutation is an ATP1-111 mutation comprising SEQ ID NO:1 or SEQ ID NO:2.

5. A transgenic non-respiring yeast having a mitochondrial gene removed from mitochondrial DNA of the non-respiring yeast while leaving the rest of the yeast mitochondrial genome intact, wherein the mitochondrial gene removed is chosen from the group comprising COX1, COX2, COX3, or COB.

6. The transgenic non-respiring yeast of claim 5, wherein said transgenic non-respiring yeast has an ethanol production rate between 5% and 25% greater than a yeast containing an intact mitochondrial genome.

7. The transgenic non-respiring yeast of claim 5, wherein said transgenic non-respiring yeast has a doubling time equal to a yeast containing an intact mitochondrial genome.

8. The transgenic non-respiring yeast of claim 5, wherein said transgenic yeast further comprises a dominant mitochondrial ATP synthase gene mutation stably integrated into the transgenic non-respiring yeast.

9. The transgenic non-respiring yeast of claim 8, wherein the dominant mitochondrial ATP synthase gene mutation is an ATP1-111 mutation comprising SEQ ID NO:1 or SEQ ID NO:2.

10. A method for enhanced fermentation of plant material through the genetic modification of transgenic non-respiring yeast comprising:
introducing a dominant mitochondrial ATP synthase gene mutation into the yeast;
removing the mitochondrial DNA from the yeast; and
producing a transgenic non-respiring yeast comprising a dominant mitochondrial ATP synthase gene mutation and lacking mitochondrial DNA, wherein the transgenic non-respiring yeast is capable of fermenting plant material.

11. The method of claim 10, wherein the dominant mitochondrial ATP synthase gene mutation is a ATP1-111 mutation comprising SEQ ID NO:1 or SEQ ID NO:2.

12. The method of claim 11, further comprising introducing a DNA construct into a transgenic non-respiring yeast lacking a mitochondrial DNA, comprising:
stably integrating a DNA construct into a vector;
stably integrating the vector into a non-respiring yeast;
removing the selectable marker from the construct; and
identifying a recombinant yeast that lacks the selectable marker but contains the dominant mitochondrial ATP synthase gene mutation;
wherein the DNA construct comprises the dominant mitochondrial ATP synthase gene mutation operably linked to a selectable marker.

13. A DNA construct for enhanced yeast fermentation of plant material through the genetic modification of transgenic non-respiring yeast comprising:
a dominant mitochondrial ATP synthase gene mutation and a selectable marker wherein the dominant mitochondrial ATP synthase gene mutation is operably linked to the selectable marker.

14. The DNA construct of claim 13, wherein the dominant mitochondrial ATP synthase gene mutation is an ATP1-111 mutation comprising SEQ ID NO:1 or SEQ ID NO:2.

15. The DNA construct of claim 13, wherein the dominant mitochondrial ATP synthase gene mutation has a first repetitive DNA sequence operably linked immediately 5′ to the dominant mitochondrial ATP synthase gene mutation and a second repetitive DNA sequence operably linked immediately 3′ to the dominant mitochondrial ATP synthase gene mutation.

16. The DNA construct of claim 15, where the first repetitive DNA sequence is SEQ ID NO:3 and the second repetitive DNA sequence is SEQ ID NO:4.

17. The DNA construct of claim 15, where the selectable marker is operably linked to a third repetitive DNA sequence 5′ to the selectable marker, wherein the selectable mark is also operably linked to the third repetitive DNA sequence 3′ to the selectable marker.

18. The DNA construct of claim 17, wherein the third repetitive DNA sequence is SEQ ID NO:5.

19. A transgenic non-respiring yeast having a DNA construct stably integrated into the transgenic non-respiring yeast under conditions suitable for expression of the DNA construct in a transgenic non-respiring yeast, wherein the DNA construct comprises a dominant mitochondrial ATP synthase gene mutation and a selectable marker and wherein said transgenic non-respiring yeast lacks mitochondrial DNA.

20. The transgenic non-respiring yeast of claim 19, wherein the dominant mitochondrial ATP synthase gene mutation is an ATP1-111 mutation comprising SEQ ID NO:1 or SEQ ID NO:2.

21. The transgenic non-respiring yeast of claim 19, wherein said transgenic non-respiring yeast has an ethanol production rate between 5% and 25% greater than a yeast not comprising a dominant mitochondrial ATP synthase gene mutation stably integrated into the yeast.

22. The transgenic non-respiring yeast of claim 19, wherein said transgenic non-respiring yeast has a doubling time equal to a yeast not comprising a dominant mitochondrial ATP synthase gene mutation stably integrated into the yeast.

23. The transgenic non-respiring yeast of claim 19, wherein said non-respiring yeast lacks a CAT5 nuclear gene.

24. The transgenic non-respiring yeast of claim 23, wherein said transgenic non-respiring yeast has a doubling time equal to a yeast not comprising a dominant mitochondrial ATP synthase gene mutation stably integrated into the yeast and comprises a CAT5 nuclear gene.

25. A method for enhanced fermentation of plant material through the genetic modification of non-respiring yeast comprising:
conducting a first transformation to delete a first copy of a CAT5 gene;
conducting a second transformation to delete a second copy of a CAT5 gene; and
isolating the non-respiring yeast strain bearing said deletion of said first copy of said CAT5 gene and said deletion of second copy of said CAT5 gene, wherein said non-respiring yeast is capable of fermentation of plant material.

26. A transgenic non-respiring yeast, wherein said transgenic non-respiring yeast comprises at least one CAT5 gene deletion.

27. The transgenic non-respiring yeast of claim 26, wherein a first copy of said CAT5 gene has been deleted from said transgenic non-respiring yeast and said second copy of said CAT5 gene has been deleted from said transgenic non-respiring yeast.

28. The transgenic non-respiring yeast of claim 26, wherein said transgenic non-respiring yeast has a doubling time equal to a yeast comprising a CAT5 nuclear gene.


SEQUENCE LISTING (9 sequences)

SEQ ID NO: 1 - protein, 545 aa, Saccharomyces cerevisiae
MLARTAAIRSLSRTLINSTKAARPAAAALASTRRLASTKAQPTEVSSILEERIKGVSDEANLNETGRVLAVGDGIARVFGLNNIQAEELVEFSSGVKGMALNLEPGQVGIFLFGSDRLVKEGELVKRTGNIVDVPVGPGLLGRVVDALGNPIDGKGPIDAAGRSRAQVKAPGILPRRSVHEPVQTGLKAVDALVPIGRGQRELIIGDRQTGKTAVALDTILNQKRWNNGSDESKKLYCVYVAVGQKRSTVAQLVQTLEQHDAMKYSIIVAATASEAAPLQYLAPFTAASIGEWFRDNGKHALIVYDDLSKQAVAYRQLSLLLRRPPGREAYPGDVFYLHSRLLERAAKLSEKEGSGSLTALPVIETQGGDVSAYIPTNVISITDGQIFLEAELFYKGIRPAINVGLSVSRVGSAAQVKALKQVAGSLKLFLAQYREVAAFAQFGSDLDASTKQTLVRGERLTQLLKQNQYSPLATEEQVPLIYAGVNGHLDGIELSRIGEFESSFLSYLKSNHNELLTEIREKGELSKELLASLKSATESFVATF

SEQ ID NO: 2 - DNA, 1638 bp, Saccharomyces cerevisiae
atgttggctcgtactgctgctattcgttctctatcgagaactctaattaactctaccaaggccgcaagacctgccgctgctgctttggcttccaccagaagattggcttccaccaaggcacaacccacagaagtttcctccatcttagaggaaagaattaagggtgtgtccgacgaggccaatttgaacgaaactggtagagttcttgcagtcggtgatggtattgctcgtgtttttggtttgaacaacattcaggctgaagaattggtcgagttctcctctggtgttaaaggtatggctttgaacttggagcctggtcaagtcggtatctttcttttcggttccgatagactggttaaagaaggtgaattggtcaagagaaccggtaatattgttgatgtcccagtcggtccaggccttttgggtagagttgtcgacgctttaggtaaccctattgatggtaaaggtcctattgacgctgccggtcgttcaagagctcaagtcaaagcaccaggtattttgccaagaagatctgtccatgaaccagttcaaaccggtttgaaagccgttgacgccttggtccctatcggtagaggtcaaagagagttgattattggtgatcgtcaaacaggtaagactgctgtcgccttagacaccatcttgaatcaaaagagatggaataacggtagtgacgaatccaagaaactttactgtgtttacgttgccgttggacaaaaaagatctaccgttgctcaattggtccaaactttggaacaacatgacgccatgaagtactctattattgttgcagctactgcatctgaagccgctcctctacaatacttggctccatttactgccgcatccattggtgaatggttcagagataatggaaagcacgctttgatcgtctatgacgatttgtccaagcaagccgtggcataccgtcaattatctttgttgttgagacgtcctcctggtcgtgaagcctaccctggtgatgtcttttacttgcattcaagattgctagaaagagccgctaagctttctgaaaaggaaggttctggttctttaactgctttgcctgttattgaaacccaaggtggtgatgtctccgcttatattccaaccaatgttatttccattaccgatggtcaaattttcttggaagctgaattattctacaagggtatcagacctgccattaacgttggtttgtccgtttctcgtgtcggttccgctgctcaagttaaggctttgaagcaagtcgctggttccttgaaattgtttttggctcaatacagagaagtcgctgcttttgctcaattcggttccgatttagatgcctccaccaagcaaactttggttagaggtgaaagattgactcaattgttgaagcaaaaccaatattctcctttggctacagaagaacaggttccattgatttatgccggtgttaatggtcatttggatggtattgaactatcaagaattggtgaatttgagtcctcctttttgtcctatctaaaatccaatcacaatgagcttttgaccgaaattagagaaaagggtgaattgtctaaagaattgttggcatctctaaagagtgctactgaatcatttgttgccactttttaa

SEQ ID NO: 3 - DNA, 200 bp, Saccharomyces cerevisiae
aactgatttctcatatattcccaaacaggcatatatactcgacgtcaagaaagaaaagaaaagaaaaccctcataaaaaatataatcgagaagtttttttcctcatcgcgaaccattagtataacagattgatcgttcagctctcataactatcgcaagaacagtaacaaaataaataaaaaaaacacgcacatataata

SEQ ID NO: 4 - DNA, 200 bp, Saccharomyces cerevisiae
tgtgaactaaaaaaataaaaatgaatataaggtacgtctcaaaaagaaatgtaaatatagaaattttaaaaaaaaaaacgaaaaaaaacataactaaatttaaagtgcagccaaacaataaccctgaaaaatctaaatatcttagaatttttttattttgattattatatattattattattcttatggtaaataatgcc

SEQ ID NO: 5 - DNA, 200 bp, E. coli
ctgtggcattaaaattaatcttcacacccagcgcctgatcgcgatggcagaaaacatgccgattgatattctgcgcgtgcgtgacgacgacattcccggtctggtaatggatggcgtggtagaccttgggattatcggcgaaaacgtgctggaagaagagctgcttaaccgccgcgcccagggtgaagatccacgctact

SEQ ID NO: 6 - DNA, 20 bp, Saccharomyces cerevisiae
ccatctttcccattgacgtt

SEQ ID NO: 7 - DNA, 20 bp, Saccharomyces cerevisiae
cttgcaggcgatatttcctt

SEQ ID NO: 8 - DNA, 26 bp, Saccharomyces cerevisiae
cggattggtacctttggatccagagc

SEQ ID NO: 9 - DNA, 26 bp, Saccharomyces cerevisiae
gtgggatccttttctagaagggttat